(19) United States
(12) Patent Application Publication
(10) Pub. No.: US 2011/0172735 A1
(43) Pub. Date: Jul. 14, 2011

(54) SYSTEMS AND METHODS FOR THERAPY

(76) Inventor: Pooja Johari, San Diego, CA (US)

(21) Appl. No.: 12/981,576

(22) Filed: Dec. 30, 2010

Related U.S. Application Data
(60) Provisional application No. 61/291,543, filed on Dec. 31, 2009.

(51) Int. Cl. A61N 1/00

(52) U.S. Cl. 607/48

(57) ABSTRACT

An electrotherapy stimulation device is provided. In an embodiment the electrotherapy stimulation device may include a cranial electrical stimulation module with a pulse generator system, a processor, and a memory. The processor can be configured to control the pulse generation system and the memory can be configured to store instructions that cause the processor to cause the pulse generator to generate cranial stimulation waveforms. The electrotherapy stimulation device may also include a body stimulation module that is coupled to the cranial stimulation system and configured to operate in conjunction with it. One example body stimulation module is an electrical muscle stimulation module. Additionally, the electrotherapy stimulation device may be used to stimulate a patient's mind and body by following a protocol that can including predetermined e-yoga, power vector, classical sequence, dynamic rotation, micro-massage, and body care.

### CROSS-REFERENCE TO RELATED APPLICATIONS

Filed in pursuant on the provisional Appl. No. 61/291,543

### FIELD OF THE INVENTION

This invention generally relates to systems and methods for medical therapy devices and more particularly, some embodiments relate to systems and methods for electrotherapy stimulation.

### BACKGROUND

Various forms of bio-electrical or other types of therapy are known throughout the medical community. These can include cranial electrotherapy, vacuum therapy, and ultrasound therapy, as well as other types. The therapies may be used to relieve a number of patient health issues. For example, cranial electrotherapy has been used to potentially provide relief from insomnia, depression, anxiety, drug addiction, and cognitive dysfunction.

The therapy regimes have been used to treat various symptoms, conditions, and aliments. New treatment regimes and combinations of treatment regimes and protocols may be used to potentially provide different patient benefits or increased patient benefits such as muscle strengthening, decreased fat volume, improved circulation, stress relief, relaxation, ATP predication, or increased energy.

### SUMMARY

An electrotherapy stimulation device is provided. In an embodiment the electrotherapy stimulation device may include a cranial electrical stimulation module with a pulse generator system, a processor, and a memory. The processor can be configured to control the pulse generation system and the memory can be configured to store instructions that cause the processor to cause the pulse generator to generate cranial stimulation waveforms.

In some embodiments, the electrotherapy stimulation device may also include a body stimulation module that is coupled to the cranial stimulation system and configured to operate in conjunction with it. One example body stimulation module is an electrical muscle stimulation module. The electrical muscle stimulation module can provide a variety of waveforms to stimulate the abdominal muscles, buttocks, or other muscle areas within a patient's body.

Other example body stimulation modules can include microcurrent therapy, laser treatment, micro-massage, massage, acupuncture, or other bodywork. Additionally, multiple types of body work might be combined in a single dual therapy system, for example, electrical muscle stimulation and massage could be combined together in some example systems. Such a system might provide electrical stimulation to the body followed by a massage. Additionally, the body work might be provided by a module such as the electrical muscle stimulation module, a massage module, microcurrent module, etc. In some cases, however, a method may include bodywork performed by a person in conjunction with cranial electrical stimulation. It will be understood that, in some examples, an electrical muscle stimulation module may also be able to provide microcurrent therapy.

In some embodiments the electrotherapy stimulation device may be used to stimulate a patient's mind and body by following a protocol that can include predetermined e-yoga, power vector, classical sequence, dynamic rotation, micromassage, and body care. Such a sequence may include multiple sessions with different treatments or combinations of treatments performed as the treatments progress.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a block diagram illustrating an example dual therapy system in accordance with an embodiment;

FIG. 2 is a block diagram illustrating an example body stimulation module in accordance with the embodiment illustrated in FIG. 1;

FIG. 3 is a graph illustrating an example power vector waveform in accordance with various embodiments of the systems and methods described herein;

FIGS. 4 and 5 are diagrams illustrating stimulation patterns in accordance with various embodiments of the systems and methods described herein;

FIG. 6 is a diagram illustrating a series of stimulations in a treatment waveform in accordance with the systems and methods described herein; and

FIG. 7 is a diagram illustrating placement of electrodes and patterns of signal movements between electrodes in accordance with the systems and methods described herein.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale. The words “including,” “has,” and “having,” as used herein, including the claims, shall have the same meaning as the word “comprising.”

FIG. 1 is a block diagram illustrating an example dual therapy system 100 in accordance with one embodiment of the systems and methods described herein. Dual therapy system 100 includes a mind stimulation portion and a body stimulation portion. The mind stimulation portion can be a cranial electrical stimulation (CES) module 102.

CES module 102 may provide treatment be transmitting small electrical current pulses across a patient's head. In some embodiments, the current used in conjunction with CES may be between 0 and 3 mA. Generally CES might sometimes be used for anxiety, depression, insomnia, drug addiction, or other patient issues. CES is also sometimes referred to as “electro-sleep therapy,” “neuro-electric therapy,” or “transcranial electrotherapy.” The CES module 102 can be used to perform “e-Yoga.” E-Yoga involves passing microcurrents of electricity across the brain.

The CES module can be used in conjunction with body stimulation provided by a body stimulation module 104. In the illustrated embodiment of FIG. 1 body stimulation is provided to the patient by the body stimulation module 104 as part of a larger dual therapy system 100. It will be understood that, in some embodiments, however, body stimulation may be provided by a separate stand-alone system. In other words, some embodiments of the systems and methods described herein relate to stand-alone body stimulation modules, waveforms for body stimulation modules, or both, while other embodiments of the systems and methods described herein relate to dual therapy systems. Generally, features and waveforms of body stimulation module 104 can be applied to stand-alone body stimulation modules. Additionally, in some embodiments, non-device based body stimulation can be used. For example, a massage might be performed by a massage module or a person can perform a massage.

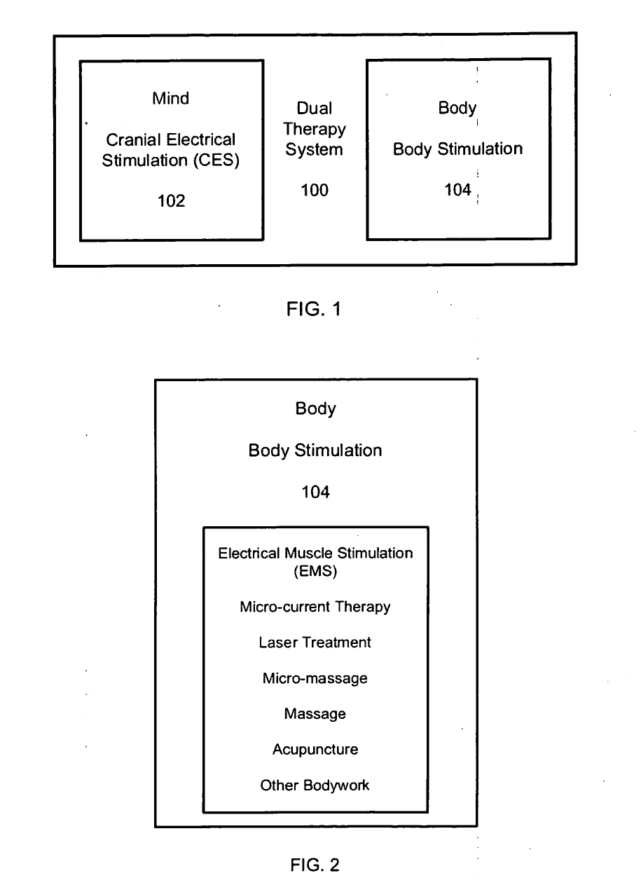

FIG. 2 is a block diagram illustrating an example body stimulation module in accordance with the embodiment illustrated in FIG. 1. Body stimulation can include, but is not limited to, electrical muscle stimulation (EMS), microcurrent therapy, laser treatment, micro-massage, massage, acupuncture, or other bodywork. As illustrated in FIG. 1, various embodiments of the systems and methods described herein may provide the body stimulation using a module within the dual therapy system 100. For example, an EMS module may be combined with a CES module to provide multiple types of therapy to a patient. Similarly, other modules such as Microcurrent modules, laser treatment modules, micro-massager modules, massage modules, automated acupuncture modules, or other automated bodywork modules may be combined with CES. Additionally, multiple body stimulation modules 104 might be combined with one or more CES modules 102. For example, an EMS module and a laser treatment module might both be combined with a CES module 102.

As discussed above, while some embodiments of the systems and methods described herein may combine CES with device-based treatments, other embodiments may use a CES device in combination with treatments that can sometimes be non-device based treatments, such as massage, acupuncture, or other body work.

Such a device may provide various EMS waveforms such as the waveforms discussed herein. In some embodiments of the systems and methods described herein these waveforms, generated by the EMS module may to provide lymphatic drainage or body contouring for a patient. Treatments for other aliments, symptoms, or patient issues may also be possible using a dual therapy system 100, stand alone body stimulation, or the waveforms described herein.

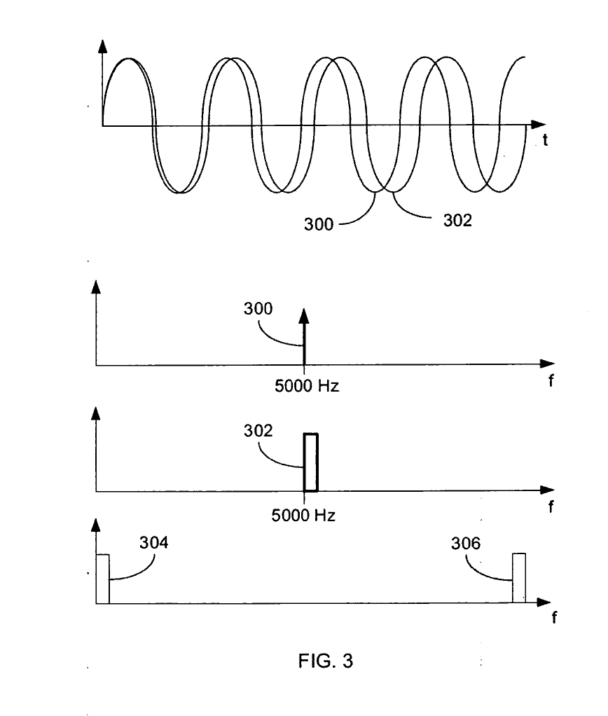

The waveforms can include a power vector waveform, a sequential stimulator waveform, dynamic rotation, and micromassage. An example power vector waveform that can be used in conjunction with an EMS module, either within a system 100 or in a standalone system are illustrated in FIG. 3. In one embodiment a power vector wave form may be generated using two medium-frequency, sinusoidal signals 300 and 302 that can intersect deep within the tissues of a patient to generate a third signal 304. The third signal is a combination of the two signals.

For example, in one embodiment the first signal 300 may be generated at 5000 Hz. The second signal 302 can sweep between 5000 Hz to 5299 Hz. In the time domain graph of FIG. 3 the first signal 300 is at 5000 Hz and the second signal 302 is at 5299 Hz, the high end of the frequency sweep. The frequency domain graphs illustrate the first signal 300 at 5000 Hz, the second signal 302 sweeping between 5000 Hz and 5299 Hz as indicated at reference character 302. Note that the figures are not to scale.

Signals 300 and 302 can combine to generate a third signal 304, which varies between dc (0 Hz) and 288 Hz. (A high frequency signal 306 at approximately 10 kHz, which also varies up to 10,288 Hz may also be generated as a result of the combination. The higher frequency signal might not provide the desired therapy for the patient, however, because the higher frequency signal may not propagate well through the patient's body.)

In one embodiment, sweeping can entail increasing frequency from 5000 Hz to 5299 Hz and the starting the sweep over at 5000 Hz. In another embodiment, the system may sweep up from 5000 Hz to 5299 Hz and then sweep down from 5299 Hz to 5000 Hz. Yet another embodiment may include both types of sweeps or other possible sweeping patterns.

It will be understood that other signal frequencies might also be used in dual therapy systems or EMS systems. For example a signal from approximately 2500 Hz to 6000 Hz might be used for the first and second signals, with the second signal varying by 0 to 288 Hz or more. At the lower frequency end (approximately 2500 Hz) the signals may increase muscle bulking, which is not desired in some embodiments. At the higher frequency end (approximately 6000 Hz) the signals may not propagate through the body as desired. It will be understood, however, that the systems and methods described herein might be combined with higher or lower frequencies than these described based on other desired results, e.g., muscle bulking; different body types which might propagate signals differently, etc.

Other frequency variations may also be used, including a second signal that varies by an amount greater than 288 Hz, a second signal that combines with the first to generate a third signal that does not include a dc (0 Hz component), or other variations. Additionally, in some embodiments, both the first and second signals may be varied to generate a third signal.

The illustrated embodiment of the power vector can include an IFC with an inbuilt 100% scan at 5000 Hz. The sweep frequency can be from 0 to 299 Hz, 1 to 299 Hz, or other sweep frequencies. Additionally, the sweep time can be 10 second up from 0 to 299 Hz and 10 seconds down 299 Hz to 0 Hz. The output may be provided to the patient using one or more pairs of electrodes. In one embodiment twelve electrodes (six pairs) might be used. These electrodes can be used for treatment to various body parts, including, for example the waistline, buttocks, or thighs.

The signals can cause small currents to flow within the tissues of the patient. The current may radiate from deep within the tissues to the surface of the patient's body stimulating the body and generally generating torsional movements within the patient's muscles. These movements may stimulate fat cells and initiate a decrease in fat volume.

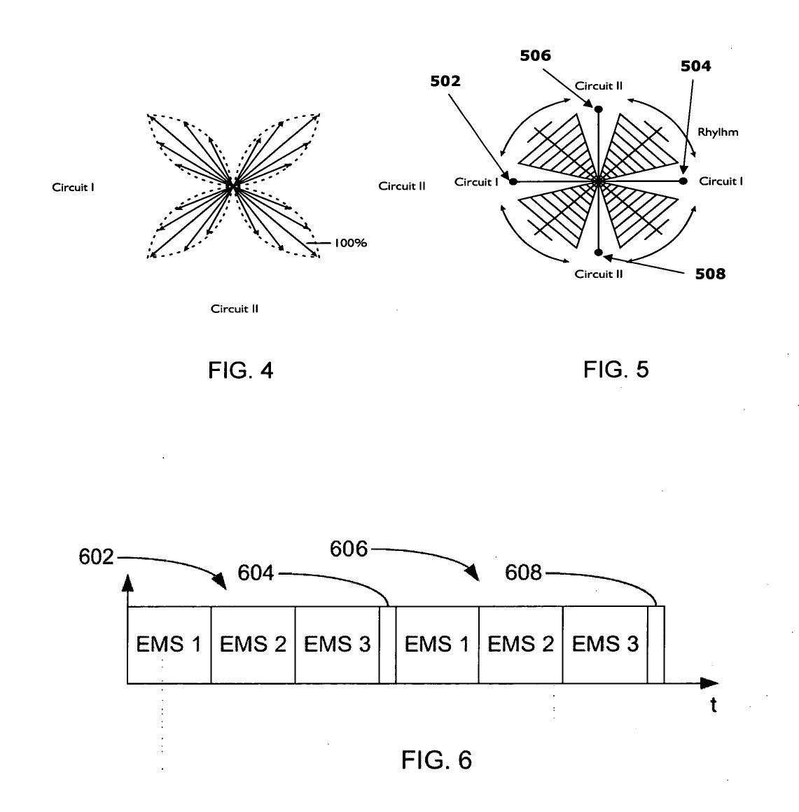

In one embodiment, the area of maximum stimulation may form a “four-leaf clover” shape. As the current is increased this four leaf clover may expand to envelop a larger and larger area. Example stimulation patterns are illustrated in FIGS. 4 and 5.

Circuit I is formed between two electrodes 502 and 504 placed on a patient's body. These electrodes 502 and 504 are energized such that current flows between them. Similarly, circuit II is formed between two electrodes 506 and 508 placed on a patient's body such that current flows between them when the electrodes are energized. The pattern illustrated in FIG. 4 may be generated using the same or a similar electrode patter at lower signal intensity.

FIG. 6 is a diagram illustrating an example series of stimulations in a treatment waveform in accordance with the systems and methods described herein. A first series of stimulations 602 may include three treatment sequences, EMS1, EMS2, and EMS3.These sequences 602 can be followed by a rest period 604 and then repeated 606 and followed by another rest period 608. The sequences EMS1, EMS2, and EMS3; followed by the rest period can be repeated according to a treatment protocol; however, it will be understood by those of skill in the art that other sequences may used.

In one embodiment the total sequence time can be 2 minutes 30 seconds with a 30 second rest period after the sequence. Accordingly, each sequence, EMS1, EMS2, and EMS3, may be 50 seconds long. In other embodiments, however, the length of each sequence EMS1, EMS2, and EMS3 are not required to be equal.

The sequences may be applied to a patient using multiple electrodes, e.g., 3 outputs with 12 electrodes. These electrodes may be attached to a patient to provide stimulation to a patient's abs, waistline, buttocks, or thighs; as well as other locations.

Sequence EMS1 can be a symmetrical biphasic square wave with a pulse width of 200 microseconds, a pulse rate of 100 Hz, and a sweep frequency of 40 to 100 Hz. The sweep time may be a two second sweep up increasing in frequency followed by a two second sweep decreasing in frequency. Sequence EMS2 can be a symmetrical biphasic square wave with a pulse width of 350 microseconds and the pulse rate of 30 Hz. Sequence EMS3 can be a symmetrical biphasic square wave with a pulse width of 350 microseconds and a pulse rate of 100 Hz.

It will be understood that while biphasic square waves are discussed with respect to EMS1, EMS2, and EMS3, other embodiment may include a square wave or other signal with a dc offset. Accordingly, the signal may have a voltage that changes between 0 volts and a positive voltage V or between 0 volts and a negative voltage −V. Alternatively, the signal may be dc offset such that the signal has a voltage that changes between V1 volts and V2 volts, where V1 may be negative, V2 may be positive, and the magnitudes of each might not be equal. Depending on the voltage of the dc offset, however, V1 and V2 may both be positive or both be negative.

Contractions are times when a signal is applied. Relaxations are times when no signal is applied. Contractions and relaxations can occur within a sequence. The relaxations within a sequence may be in addition to a “rest” at the end of the EMS1, EMS2, and EMS3 series. Each of the EMS1, EMS2, and EMS3 sequences can include a contraction time of 6 seconds followed by a relaxation time of four seconds. Additionally, a ramp up of 4 seconds and a ramp down of four seconds may be included between relaxation and contraction times.

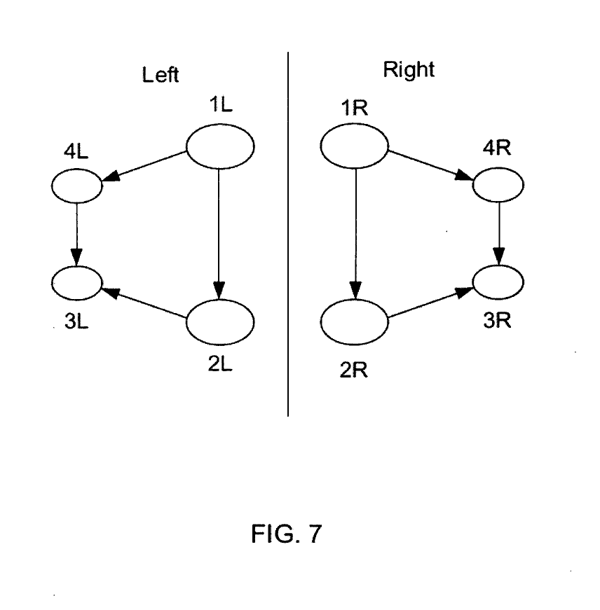

FIG. 7 is a diagram illustrating placement of electrodes and patterns of signal movements between electrodes in accordance with the systems and methods described herein.

As illustrated in FIG. 7, dynamic rotation can be performed, for example, on a patient's abdominal region. The left and right sides, which may be from the perspective of the person performing the treatment or the person being treated, can be operated independently from each other. For example, left electrodes interact with other left electrodes, while right electrodes interact with right electrodes. It will be understood, however, that some leakage of current flow might occur between left and right and that in other embodiments, electrodes on opposite sides might interact with each other. (Leakage currents might occur with respect to other waveforms as well.)

One example treatment may include four sequences. The first sequence can provide stimulation between 1L and 2L and stimulation between 1R and 2R. These stimulations can occur at the same time. The second sequence can provide stimulation between 3L and 4L and stimulation between 3R and 4R. These stimulations can also occur at the same time.

The third and fourth sequences can provide stimulation between multiple electrodes on the same side at the same time. For example, in the third sequence, stimulation can be provided between 1L and 3L while stimulation is also being provided between 2L and 4L. The third sequence can also include stimulation between 1R and 3R at the same time as stimulation is being provided between 2R and 4R. Additionally, all of these third sequence stimulations may occur at the same time. The fourth sequence can include stimulation between 1L and 2L while stimulation is also being provided between 3L and 4L. The fourth sequence can also include stimulation between 1R and 2R at the same time as stimulation is being provided between 3R and 4R. Additionally, similarly to the third sequence, all of these fourth sequence stimulations may occur at the same time.

In some embodiments each sequence can be seven seconds long. A rest period of five seconds can be included after sequence 4. Accordingly, the series of sequences may be 32 seconds long. The dynamic rotation series can include multiple four sequence series and rest periods.

In some embodiments, the dynamic rotation series can use a “premod” signal. The premod signal can be 5000 Hz with a sweep frequency from 40 to 80 Hz and a sweep time of two seconds increasing from 40 Hz to 80 Hz and two seconds decreasing from 40 Hz to 80 Hz. Various embodiments use two outputs that drive a total of eight electrodes. Some embodiments may use other sweep frequencies, such as 40 to 5000 Hz or preferably 50 to 100 Hz.

After the various other treatments described above, cool down and body care treatments can occur. These treatments can include microcurrent therapy, skin tightening treatments, a rinse to remove the seaweed gel, hydration treatments, etc. Additionally, aftertreatment measurements, photos, or both may be taken. (These may be compared to pretreatment measurements and photos, if they were taken.)

Microcurrent therapy can include a symmetrical biphasic square wave with a pulse width for a 50% duty cycle. The frequency of the square wave can be swept between 20 Hz and 150 Hz with sweep times of 10 seconds increasing from 20 Hz to 150 Hz and 10 seconds decreasing from 150 Hz down to 20 Hz. It will be understood that, as discussed above, while a biphasic square wave is discussed in the example, other embodiment may include a square wave or other signal with a dc offset. Accordingly, the signal may have a voltage that changes between 0 volts and a positive voltage V or 0 volts and a negative voltage −V. Alternatively, the signal may be dc offset such that the signal may have a voltage that changes between V1 volts and V2 volts, where V1 may be negative, V2 may be positive, and the magnitudes of each might not be equal. Depending on the voltage of the dc offset, however, V1 and V2 may both be positive or both be negative.

Some embodiments of the systems and methods described herein may a standard protocol that is a minimum of six treatments of approximately 60 minutes each. It will be understood, however, that other treatment protocols may be used. These other protocols may include longer or shorter treatment time, a fewer or greater number of total treatments, or some combination of these. In some cases the systems that implement the treatment protocol may operate with minimal intervention, e.g., for initial set up, periodic monitoring, etc.

In one example, six treatment sessions are used. The protocol has a first session which can include e-yoga treatment, power vector treatment, micro-massage treatment, and body care. A second session can include e-yoga treatment, classical sequence treatment, micro-massage treatment, and body care. A third session can include e-yoga treatment, classical sequence treatment, micro-massage treatment, and body care. Sessions four through six can include e-yoga treatment, dynamic rotation treatment, micro-massage treatment, and body care. Other example protocols may include six to ten sessions or more. It will also be understood that other protocols may also be used with the systems and methods described herein. These other protocols may include different combinations of eyoga treatment, a power vector treatment, classical sequence treatment, dynamic rotation treatment, micro-massage treatment, and body care

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. An electrotherapy stimulation device comprising:
a cranial electrical stimulation module including:
a pulse generator system,
a processor configured to control the pulse generation system, and
a memory configured to store instructions that cause the processor to cause the pulse generator to generate cranial stimulation waveforms;
a body stimulation module, coupled to the cranial stimulation system and configured to operate in conjunction with the cranial stimulation system.

2. The electrotherapy stimulation device of claim 1, wherein the body stimulation module comprises an electrical muscle stimulation module.

3. A method of mind and body stimulation comprising:
connecting an electrotherapy stimulation device to a patient, the electrotherapy device comprising:
a cranial electrical stimulation module including:
a pulse generator system,
a processor configured to control the pulse generation system, and
a memory configured to store instructions that cause the processor to cause the pulse generator to generate cranial stimulation waveforms; and
a body stimulation module, coupled to the cranial stimulation system and configured to operate in conjunction with the cranial stimulation system, the body stimulation module comprising an electrical muscle stimulation module;
stimulating the patient's mind using the mind stimulation module; and
stimulating the patient's body using the electrical muscle stimulation module, wherein the mind and body stimulation follow a protocol including predetermined e-yoga, power vector, classical sequence, dynamic rotation, micro-massage, and body care.

4. A method of mind and body stimulation comprising:
connecting an electrotherapy stimulation device to a patient, the electrotherapy device comprising:
a cranial electrical stimulation module including:
a pulse generator system,
a processor configured to control the pulse generation system, and
a memory configured to store instructions that cause the processor to cause the pulse generator to generate cranial stimulation waveforms;
stimulating the patient's mind using the mind stimulation module; and
stimulating the patient's body using non-electrical muscle stimulation.
